SEQUENCE LISTING (6 sequences)

SEQ ID NO: 1 - protein, 934 aa, Streptococcus thermophilus
  ref <308>: DATABASE ACCESSION NUMBER: SWISSPROT/ Q5M6E4 (“labB”)
  ref <309>: DATABASE ENTRY DATE: 2005-02-01
  ref <313>: RELEVANT RESIDUES IN SEQ ID NO: (1)..(934)
MLLVANPQFFHNLTKEKIYKNSTFRNYVKRSITRATPFGLFSSIGVGTFSDISYPQKIGENYTKKISVSGEWLSSICMMLENEDSVLLQLHIQWNQKVLELSDKYQLSNVNYWGISEISKDLFIKKTGLLEFIQKLTYKSEVSVLELVQTIQSKSPDLEKQKILDYIRNLITNEFLFTNLRKVAISHKDLDDLTNILKDINNQRKLTEDLLQIKNSIEQYAKLELGEGISQYTEICKKMSYIFNDEKQHYLKVDLVNCCDSVLPKNLKKKMEDFVNFISKINLTKGYRNRELESFTEKFVEKYGEYVEIPIKELLDGNLGLGLPKPSLGAQGKPSSSVEEQKFLYYLSKEVFKAIKNCKKEIDISNIPLGLLYPNSDRFVANQLELYCAIKNFESQSVISVVPNTGSDMIGKSIGRFASYFPNSYISLDSQLNNVELIEFSRDSKNLNVMSAKHAHSKKLLLSYDDNDNTSIELDSLVVGVIKIDQRYKLYFRDLRTGSIVNFVTTSMLNHKSNGVFSDLARFLLTVSLEWQDNPFSVFRIIENFDFLPYIPKIKYGDIILSEEKWVLSDIDKSDLSSINQWKKDFDVPRLLYFHKADERLLVDLENDLDIQWLLKQNVDKLYFTRFEKCDGKNCEFIFGFENYQNSINHYSMQEKSVRRLTNNFYKNYVKTFSSDWIYFRLYGINSSILPELRERLLLFTDELLVEKLISDFHFVNYRDKDNGSLRLRFKINNDNNFEDLRFRITHWIDFLLESGFCFCNDVSFNLYEREIERYGGDSFTTVCERMFSIDSFLTLKLFSKKLLNDKDLLSVLHSTFLYVRLLGIPLKRLLELLKGNLTQNRYRKSFKKLFPNNTIVVKEFKEYFDYKNQFEIFNEVFREFPPIDGYFEGKNDIVYSLLHMHMNRVGIFPYREKEYLYFIMYIAEALNNYEEFT

SEQ ID NO: 2 - DNA, 2805 bp, Streptococcus thermophilus
  ref <308>: DATABASE ACCESSION NUMBER: SWISSPROT/Q5M6E4 (“labB”)
  ref <309>: DATABASE ENTRY DATE: 2005-02-01
  ref <313>: RELEVANT RESIDUES IN SEQ ID NO: (1)..(2805)
atgttgctagtggcgaaccctcaatttttccataatttaactaaagaaaaaatatataaaaattccacctttagaaactatgtaaaaagatcaattacaagagcaacgccgtttggattgtttagttcaattggtgtaggaactttttctgatatctcatatcctcaaaagattggagaaaattatacgaaaaaaatctcggttagtggagagtggctaagttcgatttgtatgatgttagaaaatgaagattctgtcttattacaactacatatacagtggaaccaaaaagtacttgaattatcagataagtatcaattaagtaatgtcaattactggggaatctctgagataagtaaggatttatttatcaagaaaactggacttttagaattcatacaaaaattaacgtataaatcggaagtcagtgtgttagaactggtacaaacaattcaaagtaaatctcctgatttagagaagcagaaaattttagattatatacgtaatttgattactaatgaatttttgtttacaaacttgcgaaaagttgcaataagccataaagatttagatgatttaaccaacattttaaaggatattaataaccaaagaaaattaaccgaggatttattacaaataaaaaatagtattgagcagtatgcaaaattagagttgggagaaggcatttctcaatatactgaaatttgtaaaaaaatgtcgtatatttttaatgatgaaaagcagcattacctgaaggtggatttagtaaattgttgcgattcggtgttgcccaaaaatttgaaaaagaaaatggaggattttgttaattttatttcaaaaattaatctgactaaaggttatcgaaacagagaattagaatcatttacagaaaaatttgttgaaaaatatggggaatacgtcgaaattccaattaaagaattattagatggtaatttaggactgggtttaccaaaaccatctttgggagctcagggaaagccatcaagtagtgtagaagagcaaaaatttttgtattatctgagtaaagaagtcttcaaagctattaaaaattgtaaaaaagagatagatatatcaaatatacctttaggattgttatatccaaattcggatagatttgttgcaaatcagttagagttatattgtgcaattaagaattttgaatctcaatctgttatatcagttgtcccaaatacagggagtgatatgataggaaagtctattggaaggtttgcctcttattttcctaattcttatatctcgttagattcacagttaaataatgtagaattaatagaattttcaagagatagtaagaatttaaatgttatgtcggcaaagcatgctcattctaaaaagttattattatcttatgatgacaatgataacacatcgattgaattagattctctggtggtgggtgttataaaaatagaccagcgatataaactatattttagagatttacgaacaggctcaattgttaattttgtaacaacatcaatgctaaatcataaatcaaatggagtatttagcgatttagcgagatttttactaacggtttctttagaatggcaagataatccgttttcagtgtttaggataattgaaaattttgattttcttccctatattcctaaaattaaatatggagatataattttaagtgaagagaagtgggttttatcagatatagataaaagtgatttatcctctattaatcaatggaaaaaagattttgatgtacctagactactgtattttcataaagctgatgagcgtctattggttgatttagagaatgatttagatatccagtggcttttaaaacaaaatgttgataaactgtattttacgcgttttgaaaaatgtgatggtaaaaattgtgagtttatatttggatttgaaaattatcaaaatagtataaatcattattctatgcaggaaaaatctgttagaagattgacaaataacttttacaagaactatgttaaaacattttcgagtgattggatctattttaggttatatggaattaattcttcaattctacctgaactaagagagaggcttcttttatttacagatgaactattggttgaaaagctaattagtgattttcattttgtaaattatagagataaggataatgggtcgcttcgattacgttttaaaataaataatgataataattttgaagatttaagatttcgaattacacattggatagatttcctacttgaaagtggtttttgtttttgtaatgatgttagttttaatttatacgagagggaaattgagagatatggtggagatagctttacaacagtttgtgagaggatgttttctattgacagttttcttaccttaaagttattttcgaagaaattattaaatgataaagatttattatctgttttgcattcgacatttttatatgttcgattattagggatcccattaaagcgattacttgagttgctgaaggggaatttgactcaaaatagatatcgtaaaagttttaaaaagttattcccaaataacacaatagtagtaaaggaattcaaagaatattttgattacaaaaatcagtttgaaatttttaatgaagtatttagagagttcccgcctattgatggatattttgaaggtaaaaatgatatagtttatagtttacttcatatgcatatgaatagagtaggcatatttccatatagagaaaaggaatatctttattttatcatgtatattgcagaggcgttgaataattatgaggaatttacttaa

SEQ ID NO: 3 - protein, 407 aa, Streptococcus thermophilus
  ref <308>: DATABASE ACCESSION NUMBER: SWISSPROT/Q5M6E3 (“labC”)
  ref <309>: DATABASE ENTRY DATE: 2005-02-01
  ref <313>: RELEVANT RESIDUES IN SEQ ID NO: (1)..(407)
MLDSDIIDRLQRAYYTENRSYLSEYPSIIIYLSYRLINCDNKEYSKLLYNRVNYYLQELLKSIKLNSTNNISLCYGFTGYVYALEFLSKNCKGYDKLLDTLETILLALTKDRLREIKSSNIVKEEYFDVIQGVSSVARYLLSKEESTSEQELLVKEILNYFADLIINEPTIYVEYMPNEKLRKRFPSGYINLGVAHGILGPLYVLALGFEKFNITKHIPSIEKGLSYYENAFFTNTIGKIIGWNGRVSDYEENEEFRFNISWCYGSLGMARVLYNIAKIIDSQKLREMAMDVFTSSIDYLNSSEILNNGICHGRSGIMLLFNLMYLDTGKTQFKAISDNLFKEIINDASNSEYIFVERDIYFRGVTFDEVIDYIDFGLLNGVSGIVITLMAQRTGNAYPLDRMLFMQ

SEQ ID NO: 4 - DNA, 1224 bp, Streptococcus thermophilus
  ref <308>: DATABASE ACCESSION NUMBER: SWISSPROT/Q5M6E3 (“labC”)
  ref <309>: DATABASE ENTRY DATE: 2005-02-01
  ref <313>: RELEVANT RESIDUES IN SEQ ID NO: (1)..(1224)
gtgttggatagtgatattattgacagattgcagagagcatattatacagaaaatagaagttacttatcagaatatccaagtataattatatatttatcctataggttgataaattgtgataataaggagtattctaagcttctctataatagagtaaattattatctacaagaattgctaaagtctattaaattgaatagcactaataatatttcactttgctatggatttacgggctatgtatatgccttggaatttttatctaaaaattgtaaaggatatgataaattactagatacacttgagacaattttattagctttaacaaaagataggctacgtgaaataaaaagctctaatattgttaaagaagaatactttgatgtaattcaaggtgtctctagtgtagctagatatttgcttagcaaagaggaatcaacttctgaacaagagttattagtaaaagaaatattaaattactttgcagacctaataattaacgagccaactatttatgtggaatatatgccaaatgaaaaattaaggaaaagatttcctagtggctatattaatctaggtgtagctcacggtatattgggaccactatacgtgttggcgttaggatttgaaaaatttaatataacaaaacacataccttcaatagaaaagggcttgagttactatgagaacgccttttttaccaatacaattggaaaaataattggttggaatggtagagtcagtgattatgaggagaatgaggaatttagatttaatataagttggtgctatggcagtcttggaatggcaagagtactatataatatagcgaaaatcattgatagtcagaaattacgagaaatggcgatggatgtatttacttcatctattgattaccttaatagcagtgagattttaaataatggaatttgtcatggacggtctggaataatgcttttgttcaatttgatgtatttagatacaggaaagacacaatttaaagcaatttctgataatttatttaaagaaattataaatgatgcttctaattctgagtatatatttgtggagcgtgacatatattttagaggcgtaacctttgatgaagttattgattatattgactttggattgctaaatggtgtttcagggatagttataactctaatggcacaaaggactggaaatgcctacccattggatagaatgctgtttatgcagtga

SEQ ID NO: 5 - protein, 291 aa, Streptococcus thermophilus
  ref <308>: DATABASE ACCESSION NUMBER: SWISSPROT/Q5M6E2 (“labT”)
  ref <309>: DATABASE ENTRY DATE: 2005-02-01
  ref <313>: RELEVANT RESIDUES IN SEQ ID NO: (1)..(291)
MRKIFKNKLYLKVLISDLISNFGDTLYFIALMTYVTEIKDSNLAISIVNISETLPILFTIFFGIIADKTLNKVKMIIKTLWIRVLLYLLVAVVMNFKPSIMIVIIASLVNLVADTLGQFENGLFYPISNRIVKKSDREETMAFRQTVVSVMNIINQSLGAFLITFFSFSHLAFINALTFAISLLIMLAIKNQINSYYDETISTSKISQFDFKASFSEITSNLKLSINHLFSIGNMKEALLVIPILNGSLAILTPLVVVDLSKDSNLTIINSAITISLLGLALFQEEYLGAL

SEQ ID NO: 6 - DNA, 876 bp, Streptococcus thermophilus
  ref <308>: DATABASE ACCESSION NUMBER: SWISSPROT/Q5M6E2 (“labT”)
  ref <309>: DATABASE ENTRY DATE: 2005-02-01
  ref <313>: RELEVANT RESIDUES IN SEQ ID NO: (1)..(876)
atgagaaaaatctttaaaaataaactatatctaaaagttctgatttcagatttgatatcaaattttggagatacattatactttatagctctcatgacctatgttacggagattaaggatagtaatttagctatttcaatcgtcaatatatcggaaactctacctattttatttacaatattttttggtatcattgcagataaaacgcttaataaagtgaaaatgattattaaaacgttatggatacgggtactgctatatttattggttgctgtggttatgaattttaaaccttctattatgatagtcataattgctagtctcgttaatcttgttgcagatacattgggacaatttgaaaatggtcttttttatccaatttctaatcgaatagtgaaaaagtcagatagagaagaaactatggcgtttaggcaaactgttgtttctgtgatgaatattataaatcaatcattgggtgcgtttttaattactttttttagtttttctcacttagcatttataaatgctctaacttttgctattagtctgttaattatgttagcaatcaaaaatcaaattaatagctattatgacgagacgatctcgacaagtaagatatcacaatttgactttaaagcttcattttctgagattacttcaaatttaaagctctcgattaaccatctatttagtatcggcaacatgaaggaagctttacttgttataccaattcttaatgggagtttggcaattctcacgccattagttgttgttgatctttctaaagattctaatttgacaattattaattcagcgattacgatctcgcttttaggattagcactgtttcaggaggaatacttgggggcactttaa

(19) United States
(12) Patent Application Publication
(10) Pub. No.: US 2013/0004616 A1
(43) Pub. Date: Jan. 3, 2013

(54) STREPTOCOCCUS THERMOPHILUS BACTERIUM

(75) Inventor: Vega Masignani, Siena (IT)

(73) Assignee: NOVARTIS AG, Basel (CH)

(21) Appl. No.: 13/583,513

(22) PCT Filed: Mar. 10, 2011
(86) PCT No.: PCT/IB2011/051014

§ 371 Date: Sep. 7, 2012

Related U.S. Application Data
(60) Provisional application No. 61/312,479, filed on Mar. 10, 2010.

(51) Int. Cl. C12N 1/21; A23C 9/12; A23C 19/08; A23C 19/072; A23C 19/076; C12N 15/74; A23C 19/06

(52) U.S. Cl. 426/36; 435/252.3; 435/471; 426/43

(57) ABSTRACT

The present invention provides an improved Streptococcus thermophilus. The invention is based on the surprising finding that a putative lantibiotic operon in Streptococcus thermophilus is required for community formation. The operon is a 6.4 kb region that comprises three open reading frames: a lantibiotic biosynthesis protein (dehydratase—SWISSPROT REF: Q5M6E4), a lantiobiotic biosynthesis protein (cyclase—SWISSPROT REF: Q5M6E3) and a lantibiotic efflux protein (permease—SWISSPROT REF: Q70C59). Inhibiting the function of this operon inhibits community formation. Streptococcus thermophilus with a reduced ability to form communities are useful in the processing, producing or manufacturing of dairy products, in particular cheese, where Streptococcus thermophilus are often used as a starter culture.

### FIELD

This invention relates to Streptococcus thermophilus (S. thermophilus) bacteria having reduced ability to form communities, in particular biofilms, and their use in industrial processes, in particular the processing and manufacture of dairy products such as cheese.

### BACKGROUND

A bacterial community is a structured collection of bacterial cells enveloped in a self-produced polymeric matrix and adherent to an inert or living surface. It is estimated that 99.9% of bacteria in nature are attached to a surface in the form of bacterial communities. A mature bacterial community on a surface is often referred to as a “biofilm”.

Bacteria within communities exhibit two fundamental characteristics: production of extracellular polymeric substance (EPS) matrix and increased resistance to antimicrobial treatment. Bacterial community development is a multistep process initiated when bacterial cells attach to a surface, proliferate, form microcolonies and extrude a complex extracellular matrix that binds cells together and to a surface, resulting in mature community formation (Sauer et al, J. Bacteriol. 2002 February; 184(4):1140-54).

The formation of an unwanted bacterial community on a surface can be very problematic. When the surface is an industrial surface, the presence of the bacteria can reduce the efficiency of the machine or plant of which the surface is a part. Further, when the surface is one used in the production of a foodstuff, the additional, and potentially more serious, problems of spoilage and contamination also exist (Flint et al, J Appl Microbiol. 1997 October; 83(4):508-17).

Streptococcus thermophilus is a Gram-positive facultative anaerobe bacterium that is commonly used as a starter culture in the production of dairy products, including yoghurt and cheese, owing to its ability to tolerate the high temperatures required for pasteurization (typically 72° C. for 15 seconds). A major problem exists when Streptococcus thermophilus forms a community, in particular a biofilm, on the surfaces of a dairy factory or plant in which dairy products are processed and produced. Such bacterial community formation threatens the quality of the dairy products.

Current methods for the prevention of bacterial community, in particular biofilm, formation in a dairy factory or plant rely on regular cleaning of the surfaces. This cleaning is time consuming (thereby reducing the productivity of the factory or plant) and often requires expensive cleaning equipment and reagents. Further, it is not possible to guarantee that cleaning will remove sufficient bacteria from the surfaces to be successful in preventing the formation of a problematic bacterial community. Accordingly, there is a need for an improved method of preventing Streptococcus thermophilus from forming communities, in particular on surfaces involved in the processing and production of dairy products.

### SUMMARY OF THE INVENTION

The present invention is based on the surprising identification of a putative lantibiotic operon in Streptococcus thermophilus that is required for community formation. Inhibiting the function of this operon inhibits community formation.

A first aspect of the invention relates to a Streptococcus thermophilus bacterium comprising a mutation in a lantibiotic operon, wherein the mutation reduces or removes the ability of the bacterium to form a community with other bacteria, on a surface.

A second aspect of the invention relates to the use of a Streptococcus thermophilus bacterium according to the first aspect in the processing, production or manufacture of a dairy product.

A third aspect of the invention relates to a method of processing, producing or manufacturing a dairy product comprising the use of a Streptococcus thermophilus bacterium according to the first aspect.

A fourth aspect of the invention relates to a method of producing a Streptococcus thermophilus bacterium having reduced or no ability to form a community compared to wild-type Streptococcus thermophilus, comprising the step of mutating a lantibiotic operon in the Streptococcus thermophilus bacterium.

### BRIEF DESCRIPTION OF THE DRAWINGS

FIG. 1 shows the effect of the lantibiotic operon on community formation of a wild type and lantibiotic negative mutant streptococcal strain. The wild-type INV200 strain and its lantibiotic deletion mutant were tested for polystyrene community formation in C-30 mM glucose media. The results represent the averages of three independent experiments. Error bars indicate the standard error of the mean. OD540, optical density at 540 nm.

FIG. 2 shows a growth curve of wild type INV200 strain and lantibiotic mutant Δlan INV200 strain. C-30 mM glucose media was inoculated with overnight culture of INV 200 strain and mutant ΔlanINV200 strain. Aliquots were removed at the times indicated to measure optical density at 600 nm (OD₆₀₀).

### DETAILED DESCRIPTION OF THE INVENTION

### Streptococcus Thermophilus

The invention relates to the Gram-positive facultative anaerobe bacterium known as Streptococcus thermophilus. Exemplary strains of Streptococcus thermophilus include CNRZ1066 and LMG18311 (which has been deposited with the American Tissue Culture Collection as ATCC No: BAA-250); these strains have been sequenced and contain the lantibiotic operon of the invention.

It has been surprisingly found that the lantibiotic operon of Streptococcus thermophilus is required for community (e.g. biofilm) formation. The downregulation of one or more ORFs in this operon, for example due to the presence of a loss-of-function mutation in the lantiobiotic operon, reduces the ability of the bacterium to form a community (e.g. biofilm). Therefore, the Streptococcus thermophilus bacterium of the invention lacks a fully-functional lantibiotic operon and has a reduced ability to form a community.

A mutation that reduces the function of a target ORF is commonly referred to as a “loss-of-function” mutation. The lantibiotic operon has two functions; a first function is to produce a lantiobiotic and a second function is to allow community formation. The function of interest to the present invention is the ability to allow community formation. Any mutation that causes a reduction in the community-forming function of the lantibiotic operon, compared to a wild-type bacterium, is within the scope of the present invention.

As used herein, the term “wild-type” refers to a Streptococcus thermophilus that does not contain a downregulated ORF, for example a loss-of-function mutation, in its lantibiotic operon and thus retains its naturally-occurring ability to form a community.

Typically, the Streptococcus thermophilus bacterium of the invention that has a reduced ability to form a community retains a viability and a growth ability similar to wild-type Streptococcus thermophilus. The Streptococcus thermophilus bacterium of the invention is therefore viable. The growth rate of the Streptococcus thermophilus bacterium of the invention is typically similar to the growth rate of a wild-type Streptococcus thermophilus bacterium, and typically has a growth rate of at least 60%, at least 70%, at least 80% or at least 90% of the growth rate of a wild-type Streptococcus thermophilus bacterium.

### Community Formation

The Streptococcus thermophilus bacterium of the invention has a reduced ability to form a community. As used herein, the term “community” is to be given its usual meaning in the art, and refers to a plurality of bacteria attached to a surface. A mature bacterial community is a structured collection of bacterial cells enveloped in a self-produced polymeric matrix and attached to a surface. A mature bacterial community on a surface is often referred to as a “biofilm”. A biofilm may thus be defined as a bacterial community embedded in an extracellular matrix and adhered to a surface.

Mature community development is a multistep process initiated when bacterial cells attach to a surface, proliferate, form microcolonies and extrude a complex extracellular matrix that binds cells together and to a surface, resulting in mature community formation. Bacteria within communities typically exhibit two fundamental characteristics: the production of extracellular polymeric substance (EPS) matrix and increased resistance to antimicrobial treatment.

The Streptococcus thermophilus bacterium of the invention has a reduced ability, or no ability, to form a community (e.g. biofilm) with other Streptococcus thermophilus bacteria. Any reduction in the ability of the bacterium to form a community is within the scope of the invention. In one embodiment, the ability to form a mature community is removed, i.e. a culture of bacteria according to the invention will not have the ability to form a recognizable community on a surface. In other typical embodiments, there is greater than a 10%, 20%, 30%, 40%, 50%, 60%, 70%, 80% or 90% reduction, compared to a wild-type bacterium, in the bacterium's ability to form a community.

Any method can be used to detect community formation. A typical method is the in vitro “crystal violet” bacterial community formation assay, wherein bacteria are inoculated at a suitable concentration (such as 1:10 or 1:20 dilution) from a frozen culture adjusted at OD 0.3, and grown for 24 h at 37° C. in 24-well polystyrene plates. The wells of the plates, containing bacterial culture, are stained after 24 h with crystal violet (CV) and the proficiency of bacterial community formation is quantified by measuring the optical density at 540 nm (OD₅₄₀) of dissolved crystal violet (CV). An example of this assay is provided below.

A bacterial community forms on a surface. This surface will typically be an abiotic surface, for example the surfaces in a dairy factory or processing plant. Typical surfaces comprise a metal, ceramic, stone and/or plastic. A typical metal used in dairy processing surfaces is steel, more typically stainless steel. An example of a typical surface is a surface in or on a heat exchanger plate, such as those found in a pasteurizer; these heat exchanger plates have been found to be particularly susceptible to the formation of a Streptococcus thermophilus community (e.g. biofilm). A further example of a typical surface is a membrane used in filtration (including microfiltration and ultrafiltration), osmosis and reverse-osmosis of dairy products, in particular milk.

### Lantibiotic Operon

Lantibiotics are peptide antibiotics with high antimicrobial activity against several Gram-positive bacteria. The term “lantibiotic” is derived from “lanthionine-containing peptide antibiotic”. Bacteria are known to produce a family of lantibiotics known as bacteriocins, which are ribosomally synthesized and usually activated by post-translational modification involving dehydration (by a dehydratase) and then cyclisation (by a cyclase) to create the active bacteriocin.

The Streptococcus thermophilus lantiobiotic operon comprises three open reading frames: a lantibiotic biosynthesis protein (dehydratase—“labB”; SWISSPROT REF: Q5M6E4 in strain LMG18311, the protein and nucleotide sequences of which are provided below as SEQ ID Nos. 1 and 2, respectively), a lantiobiotic biosynthesis protein (cyclase—“labC”; SWISSPROT REF: Q5M6E3 in strain LMG18311, the protein and nucleotide sequences of which are provided below as SEQ ID Nos. 3 and 4, respectively) and a lantibiotic efflux protein (permease—“labT”; SWISSPROT REF: Q5M6E2 in strain LMG18311, the protein and nucleotide sequences of which are provided below as SEQ ID Nos. 5 and 6, respectively). In the operon, these sequences are present in the order “5′-labB-labC-labT-3′”. The polynucleotide and polypeptide sequences from the LMG18311 strain are provided below as SEQ ID No. 1 to SEQ ID No.6. Equivalent sequences in other Streptococcus thermophilus strains will be readily identifiable. These equivalent sequences will typically have 60% or more sequence identity to the corresponding LMG18311 sequence, more typically 70% or more sequence identity to the corresponding LMG18311 sequence and yet more typically 80% or more sequence identity to the corresponding LMG18311 sequence.

The Streptococcus thermophilus bacterium of the invention has one, two or three of its lantiobiotic operon ORFs downregulated or inactivated. When two of the ORFs are downregulated or inactivated, any combination of the ORFs can be downregulated or inactivated. Typical combinations include Q5M6E4 and Q5M6E3; Q5M6E4 and Q5M6E2; and Q5M6E3 and Q5M6E2.

The terms “downregulated”, “downregulating” and “downregulation”, as used herein with reference to an ORF in the lantiobiotic operon of Streptococcus thermophilus of the invention, refer to the reduction of expression of said ORF relative to the level of expression of that ORF in the corresponding, unmodified wild-type Streptococcus thermophilus under identical conditions. Similarly, the terms “inactivated”, “inactivating” and “inactivation”, as used herein with reference an ORF in the lantiobiotic operon of Streptococcus thermophilus of the invention, refer to the complete prevention of expression of said ORF.

Typically, the downregulation or inactivation of one or more of the lantiobiotic ORFs is caused by one or more mutations that impair the bacterium's ability to form a community with other bacteria. Any mutation in the lantibiotic operon that reduces or removes the bacterium's ability to form a community is within the scope of the invention. The mutation can be an insertion, deletion or substitution and can affect one, two or three of the ORFs in the operon. Any mutation that downregulates or inactivates expression of one or more of the ORFs is within the scope of the invention. In one embodiment, one or more of the ORFS, typically two or more and more typically all three ORFs are downregulated or inactivated, typically by being “knocked out”, i.e. deleted. The deletion of the entire lantibiotic operon, i.e. of all three ORFs, creates a lantibiotic negative strain referred to herein as Streptococcus thermophilus Δlan.

Inactivation or downregulation of one, two or three of the lantibiotic operon ORFs may be carried out using any method known in the art. For example, the sequence of one, two or three of the lantibiotic ORFs may be partially or totally deleted, and additionally may be subject to allelic replacement, or may be subjected to mutation including substitution, insertion or frameshift mutation in order to inactivate the encoded protein(s). Alternative methods of gene downregulation and inactivation, such as the use of antisense RNA or siRNA in order to prevent expression of the gene sequence or transposon mutagenesis in order to inactivate the gene, may also be employed.

### Dairy Products

The Streptococcus thermophilus bacterium of the invention is useful in processing, producing or manufacturing a dairy product. A typical processing step is pasteurization and a typical production or manufacturing step is the production or manufacture of cheese or yoghurt from a precursor such as milk.

Streptococcus thermophilus bacteria are commonly used as a starter culture in the production and manufacture of dairy products, owing to their ability to tolerate the high temperatures required for pasteurization (typically 72° C. for 15 seconds). Once Streptococcus thermophilus are introduced into the dairy product processing chain, they often form communities on the equipment. These communities can be detrimental to the dairy products. It is therefore preferable for the Streptococcus thermophilus that are used in the processing, production and manufacture of dairy products to have a reduced ability to form a community.

The term “dairy product” is to be given its usual meaning, i.e. any product prepared from animal milk. Animals from which the milk can be obtained include cows, goats, sheep, buffalo, horses, yak, reindeer and camels. Dairy products include milk, cream, cheese, yoghurt, butter and intermediates or by-products thereof, such as curds and whey. Many dairy products are pasteurized, i.e. subjected to a high temperature for a short period (typically 72° C. for 15 seconds), and a typical dairy product is therefore a pasteurized dairy product.

An exemplary dairy product is a cheese. Typically, cheese is produced from the milk of cows, goats, sheep, buffalo, horses, yak, reindeer and camels. Examples of suitable cheeses include brie, caerphilly, camembert, cheddar, cheshire, cottage, dolcelatte, edam, emmental, feta, grana padano, gouda, gruyere, halloumi, jarlsberg, leerdammer, leicester, mascarpone, monterey jack, mozzarella including buffalo mozzarella, paneer, parmesan, parmigiano reggiano, pecorino, pepper jack, port-salut, provolone, red leicester, ricotta, roquefort, stilton, swiss and wensleydale cheese.

### Modes of Performing the Invention

To ascertain whether the lantibiotic operon is involved in streptococcal bacterial community formation, a mutant in the INV200 strain (S. pneumoniae) was constructed by insertional mutagenesis. Specifically an isogenic mutant was created, containing a deletion of the entire lantibiotic operon (Δlan INV 200), as confirmed by PCR. Briefly, fragments of approximately 500 bp upstream and downstream of the target gene were amplified by PCR and spliced to an antibiotic cassette (kanamycin); the PCR fragments were then cloned into pGEMt (Promega) and transformed in the appropriate S. pneumoniae strain by conventional methods.

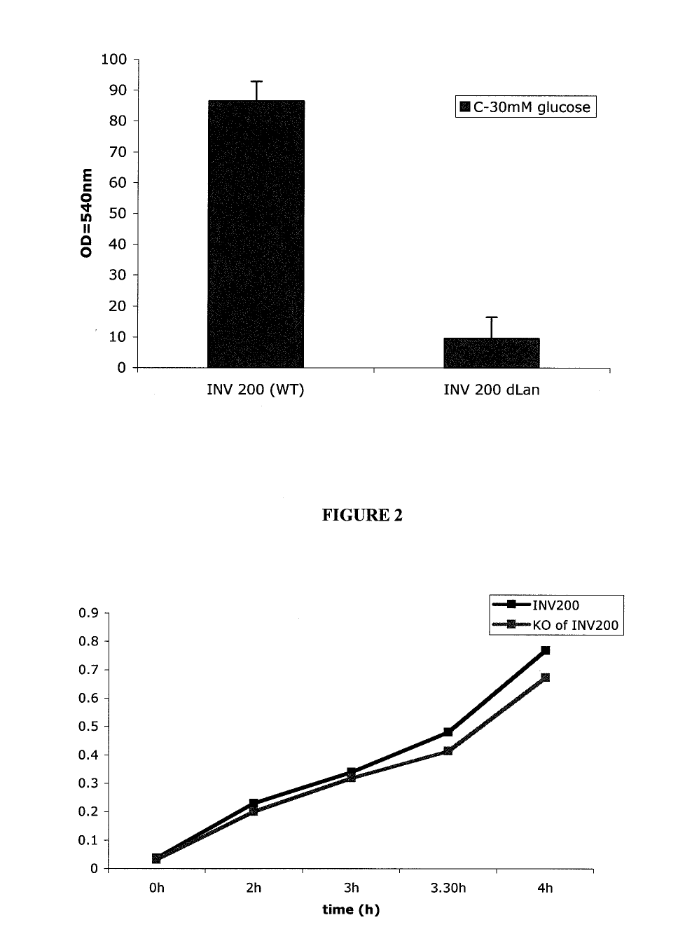

The wild type INV200 strain and its isogenic lantibiotic-negative strain were tested for development of bacterial communities. After 24 hours of growth in C-medium supplemented with 30 mM glucose, the bacterial community formation was measured as the OD₅₄₀. As shown in FIG. 1 the lantibiotic deletion resulted in a drastic reduction in community formation, indicating that the knockout mutant is defective in the production of community. Lack of community formation by the knockout strain demonstrates that development of bacterial community in wild-type strain INV200 is supported by the presence of lantibiotic operon.

In addition, to check if deletion of lantibiotic operon affects the cell growth, the growth rate in wild type INV200 strain and its lantibiotic mutant was determined. Both strains were grown in C media supplemented with 30 mM of glucose (C-30 mM) at 37° C. and monitored for growth by reading the OD₆₀₀. The deletion mutant showed a growth rate very similar to that observed for the wild type strain (FIG. 2). The almost unaltered growth rate in mutant strain suggests that deletion of the lantibiotic operon did not affect the viability and growth ability of the mutant.

In conclusion, these results clearly indicate that deletion of the lantibiotic deletion impairs bacterial community formation in streptococcus. Thus the absence of bacterial community formation in the knockout mutant demonstrates the necessity of lantibiotic operon in community development in the CC15 strain INV 200.

The data in FIGS. 1 and 2 relate to S. pneumoniae. The inventors have observed that the same lantiobiotic operon is present in S. thermophilus and it is expected that mutation of the operon in S. thermophilus will reduce the ability of the S. thermophilus to form a community. As noted above, S. thermophilus with a reduced ability to form a community are particularly useful in the dairy industry. Accordingly, it is expected that the invention can be performed using the following modes.

Creation of a Streptococcus thermophilus Mutant

An isogenic lantibiotic mutant can be made by PCR-based overlap extension and spliced to an antibiotic cassette (e.g. kanamycin); the PCR fragments can then be cloned into pGEMt (Promega) and can be transformed in the appropriate S. thermophilus strain by conventional methods.

To select the bacteria in which the target gene is replaced with the antibiotic cassette, bacteria can be plated on blood-agar plates with kanamycin (500 μg/ml). Mutants can then be confirmed by PCR.

### Detection of Bacterial Community Formation in Streptococcus Thermophilus In Vitro

A wild type Streptococcus thermophilus and its isogenic lantibiotic-negative strain can be tested for development of bacterial communities. After 24 h of growth in C-medium supplemented with 30 mM glucose, the bacterial community formation can measured as the OD₅₄₀ (as detailed below). The lantibiotic deletion should result in a significant reduction in community formation, indicating that the knockout mutant is defective in production of a community. Lack of community formation by the knockout strain will demonstrate that development of bacterial community in wild-type Streptococcus thermophilus is supported by the presence of the lantibiotic operon.

In addition, to check if deletion of the lantibiotic operon affects the cell growth, the growth rate in wild type Streptococcus thermophilus and its lantibiotic mutant can be tested. Both strains will be grown in C media supplemented with 30 mM of glucose (C-30 mM) at 37° C. and monitored for growth by reading the OD₆₀₀.

### Quantitative Bacterial Community Plate Assay

Bacterial community formation ability can be measured by determination of adhesion to a polystyrene plate. Bacteria are diluted 1:10 and 1:20 in the required culture media (e.g. those mentioned above), and 1 ml added per well of a 24 well plate (Costar, flat bottom, tissue culture treated). Plates are then incubated at 37 C. for 24 h. The medium is then removed and adherent bacteria stained with 0.2% crystal violet at room temperature for 10 min. Crystal violet is recovered with 1% SDS and proficiency of bacterial community formation is quantified by measuring the OD₅₄₀ of dissolved crystal violet. Each strain and condition should be tested in at least three independent experiments.

Data are normalized to a positive control. Additionally, sterile medium can always be included (blank control) to ensure that influence on bacterial community formation by glucose or peptone was not attributed to a nonspecific binding effect to crystal violet. The cutoff value for determining a production of community can be set at four times the negative-control value.

1. A Streptococcus thermophilus bacterium comprising a mutation in a lantibiotic operon, wherein the mutation reduces or removes the ability of the bacterium to form a community with other bacteria on a surface.

2. The Streptococcus thermophilus bacterium of claim 1, wherein the operon comprises a lantibiotic biosynthesis protein (dehydratase), a lantiobiotic biosynthesis protein (cyclase) and a lantibiotic efflux protein (permease).

3. The Streptococcus thermophilus bacterium of claim 1, wherein the mutation comprises a deletion.

4. The Streptococcus thermophilus bacterium of claim 3, wherein one or more open reading frames in the lantibiotic operon are deleted.

5. The Streptococcus thermophilus bacterium of claim 4, wherein the entire lantibiotic operon is deleted.

6. The Streptococcus thermophilus bacterium of claim 1, wherein the surface is abiotic.

7. The Streptococcus thermophilus bacterium of claim 6, wherein the surface is a metal, ceramic, stone or plastic.

8. The Streptococcus thermophilus bacterium of claim 1, wherein the community is a biofilm.

9. (canceled)

10. A method of processing, producing or manufacturing a dairy product comprising the use of the Streptococcus thermophilus bacterium of claim 1.

11. The method of claim 10, wherein the Streptococcus thermophilus bacterium is used as a starter culture.

12. The method of claim 10, wherein the dairy product is yoghurt or cheese.

13. The method of claim 12, wherein the cheese is brie, caerphilly, camembert, cheddar, cheshire, cottage, dolcelatte, edam, emmental, feta, grana padano, gouda, gruyere, halloumi, jarlsberg, leerdammer, leicester, mascarpone, monterey jack mozzarella including buffalo mozzarella, paneer, parmesan, parmigiano reggiano, pecorino, pepper jack, port-salut, provolone, red leicester, ricotta, roquefort, stilton, swiss or wensleydale cheese.

14. A method of producing a Streptococcus thermophilus bacterium having reduced or no ability to form a community compared to wild-type Streptococcus thermophilus, comprising the step of mutating a lantibiotic operon in the Streptococcus thermophilus bacterium.
